(19) United States
(12) Patent Application Publication
(10) Pub. No.: US 2012/0165840 A1
(43) Pub. Date: Jun. 28, 2012

(54) Medical needle holder

(76) Inventors: Sven Schneider, Tuttlingen (DE); Uwe Bacher, Tuttlingen (DE)

(21) Appl. No.: 13/336,676

(22) Filed: Dec. 23, 2011

(30) Foreign Application Priority Data
Dec. 23, 2010 (DE) 10 2010 055 807.9

(51) Int. Cl. A61B 17/04

(52) U.S. Cl. 606/147

(57) ABSTRACT

A medical needle holder having a shaft, a tool mounted on the distal end of the shaft, two jaw members, such that one jaw member of the tool is configured to pivot with respect to the other jaw member, a handle positioned on the proximal end of the shaft and at least two gripping members, such that the pivotable jaw member of the tool can be actuated by a movably configured gripping member of the handle.

### CROSS-REFERENCE TO RELATED APPLICATIONS

The present application claims priority of German patent application No. 10 2010 055 807.9 filed on Dec. 23, 2010.

### FIELD OF THE INVENTION

The invention relates to a medical needle holder with a shaft, with a tool positioned on the distal end of the shaft having two jaw members such that one jaw member of the tool is configured to pivot with respect to the other jaw member, and with a handle positioned on the proximal end of the shaft having at least two gripping members, such that the pivotable jaw member of the tool can be actuated by a displaceably configured gripping member of the handle.

### BACKGROUND OF THE INVENTION

Generic medical needle holders are known in various embodiments. Needles, which as a rule are curved in a sickle shape, are gripped by the tool and held by clamping between the jaw members of the tool. To align the needle, held in such a manner, in the correct position for suturing a surgical site, the operator must either manually align the needle or else move it to the required position between the jaw members of the tool by means of an additional gripping forceps.

### SUMMARY OF THE INVENTION

Consequently, it is the object of the invention to provide a medical needle holder that allows a simple positioning of the needle in the tool.

This object is fulfilled, according to the invention, in such a way that at least one portion of the non-pivotable jaw member of the tool is configured so that it can be slid in the longitudinal direction of the shaft in relation to the other jaw member of the tool, and that the sliding in the longitudinal direction of the shaft occurs by means of a power drive positioned on the handle.

Owing to the inventive configuration of the jaw members of the tool, according to which at least one portion of at least one jaw member of the tool is configured to slide in the longitudinal direction of the shaft with respect to the other jaw member of the tool, it is possible for the first time to align the position of the surgical needle held between the jaw members without utilizing an additional tool or without manual gripping. The sliding by the jaw member or partial area of a jaw member in the longitudinal direction of the shaft occurs, according to the invention, via a power drive positioned on the handle.

It is proposed, with a practical embodiment of the invention, that the slidable jaw member or slidable portion of the jaw member and the power drive should be configured as an adjusting wheel, so that a very simple actuation becomes possible with the holding hand.

According to a practical embodiment of the invention, it is proposed that the slidable jaw member or slidable portion of the jaw member and the power drive should be in operative connection with one another via an actuating element, which converts the rotary motion of the adjusting wheel into a longitudinal movement of the slidable jaw member or slidable portion of the jaw member.

To transmit the purely axial motion for actuating the pivotable jaw member, it is proposed with the invention that the pivotable jaw member of the tool and the movably configured gripping member of the handle should be in operative connection with one another via a push-pull element.

According to a first practical embodiment of the invention, it is proposed that the actuation element and the push-pull element should be positioned coaxially to one another in the shaft. The coaxial positioning of the two components constitutes a configuration that conserves space.

According to a preferred embodiment of the coaxial positioning of the actuation element and push-pull element, it is proposed with the invention that the actuation element should be configured as a hollow tube positioned in the shaft, such that the adjusting wheel and the hollow tube are in operative connection with one another via a threading and such that the slidable jaw member or the slidable portion of the jaw member forms the distal end of the hollow tube.

The push-pull element is configured in this coaxial configuration preferably as a push-pull rod positioned so that it can slide in the hollow tube.

According to a second practical embodiment of the invention, it is proposed that the actuation element and the push-pull element should be positioned parallel alongside one another in the shaft.

According to the parallel positioning of the actuation element and the push-pull element, it is proposed with the invention that the actuation element should be configured as a torsion bar positioned in the shaft, such that the adjusting wheel and the torsion bar are in a friction-locked connection with one another and such that the slidable portion of the jaw member and the distal end of the torsion bar are in operative connection with one another via a threading.

To actuate the pivotable jaw member, the push-pull element in this embodiment is preferably configured as a push-pull rod positioned in the shaft.

According to a third practical embodiment of the invention, it is proposed that the actuation element and the push-pull element should be configured as a one-piece, rod-shaped drive element positioned in the shaft, by which both the axial motion of the movable gripping member and the rotation of the adjusting wheel can be transmitted.

To transmit the axial motion from the movably configured gripping member of the handle to the pivotable jaw member of the tool, it is proposed in this one-piece configuration that the drive element should be mounted at the proximal end on the movably configured gripping member by a spherical connecting element and on the distal end in the area of the mounting on the pivotable jaw member in the outer mantle surface of the drive element a surrounding groove should be configured in which a ring is positioned that can pivot around the longitudinal axis of the drive element and on which the pivotable jaw member is mounted. The spherical configuration of the proximal end of the drive element allows a freely rotatable mounting on the movable gripping member, with simultaneous free play for transmitting axial movements.

It is further proposed with the invention that to transmit the rotary motion of the adjusting wheel into a longitudinal movement of the slidable portion of the jaw member, the drive element should be in a friction-locked connection with the adjusting wheel on the proximal end, and on the distal end in the area of the mounting on the slidable portion of the jaw member it should be in a friction-locked connection with a sheath that is mounted coaxially on the distal end of the drive element and that is connected with the slidable portion of the jaw member by a threading.

To uncouple the axial motion and the rotary motion of the drive element from one another, equalizing guides are configured according to the invention in the areas of the friction-locked connections of the drive element with the adjusting wheel as well as of the drive element with the sheath.

To configure the friction-locked connections of the drive element with the adjusting wheel, as well as of the drive element with the sheath, according to the invention driving pins are positioned in the drive element to engage the adjusting wheel and sheath with one another.

It is finally proposed with the invention that the equalizing guides should be configured as grooves that are positioned in the adjusting wheel and in the sheath and run in the axial direction and in which the driving pins are mounted.

Further properties and advantages of the invention can be seen from the appended drawings, in which various embodiments of an inventive medical needle holder are illustrated only by way of example, without restricting the invention to these embodiments.

### BRIEF DESCRIPTION OF THE DRAWINGS

FIG. 1 shows a schematic side view of an inventive medical needle holder with the jaw members in a first working position;

FIG. 2 shows a depiction according to FIG. 1, but showing the jaw members in a second working position;

FIG. 3 shows an enlarged schematic view of a first embodiment of detail III according to FIG. 1;

FIG. 4 shows an enlarged schematic view of the first embodiment of detail IV according to FIG. 1;

FIG. 5 shows an enlarged schematic view of a second embodiment of detail V according to FIG. 1;

FIG. 6 shows an enlarged schematic view of the second embodiment of detail VI according to FIG. 1;

FIG. 7 shows an enlarged schematic view of a third embodiment of detail VII according to FIG. 1; and

FIG. 8 shows an enlarged schematic view of the third embodiment of detail VIII according to FIG. 1.

### DETAILED DESCRIPTION OF THE INVENTION

The illustrated medical needle holder 1 consists essentially of a hollow shaft 2 on whose proximal end a handle 5, which comprises two gripping members 3 and 4, is positioned, such that in the illustrated embodiment the gripping member 3 is mounted on the handle 5 so that it can pivot with respect to the other gripping member 4.

On the distal end of the shaft 2 a tool 6 is positioned that, in the illustrated embodiment, consists of a jaw member 6a rigidly connected with the shaft 2 and of a jaw member 6b that can pivot around a pivot axis 7. To open and close the jaw members 6a and 6b of the tool 6 via the actuation of the pivotable gripping member 3 of the handle 3, the pivotable gripping member 3 and the pivotable jaw member 6b are in operative connection with one another via a push-pull element 8 mounted in the hollow shaft 2.

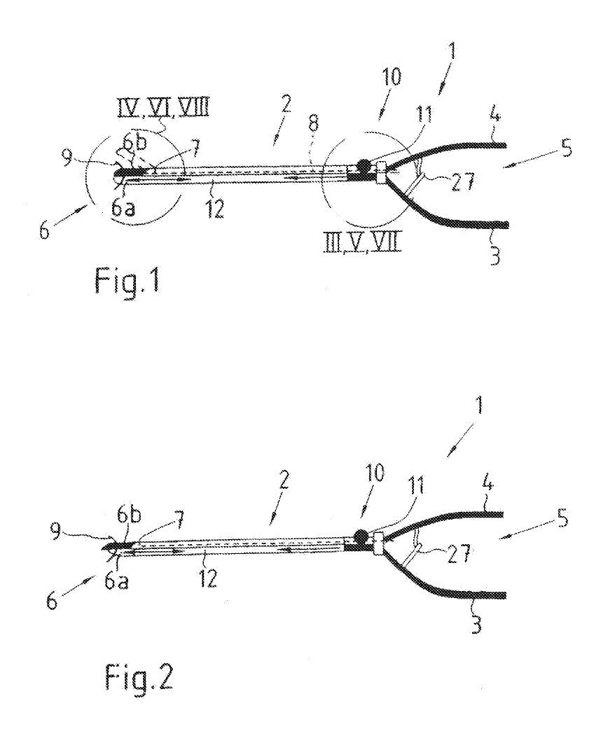

As can be seen from FIGS. 1, 2, 4, 6 and 8, the jaw members 6a and 6b of the tool 6 are not only configured in such a way that the jaw member 6b can pivot with respect to the jaw member 6a, but also the jaw member 6a or a portion 6c of the jaw member 6a is configured so that it can slide with respect to the jaw member 6b in the longitudinal direction of the shaft 2.

Alternatively to the illustrated configuration of the jaw members 6a and 6b of the tool 6, it is also possible of course that the upper jaw member 6b can be configured rigidly, that is not pivotably, and that the lower jaw member 6a can be configured pivotably. Likewise, the jaw member 6b or a portion of the jaw member 6b can be configured to slide with respect to the jaw member 6a in the longitudinal direction of the shaft 2. Another possible construction variant is for both jaw members 6a and 6b to be relatively slidable with respect to one another.

Owing to this configuration of the jaw members 6a and 6b of the tool 6, according to which at least one portion 6c of at least one jaw member 6a or 6b of the tool 6 is configured to slide in relation to the other jaw member 6b or 6a of the tool 6 in the longitudinal direction of the shaft 2, it is possible to align the position of a surgical needle 9 held between the jaw members 6a and 6b without utilizing an additional tool or without manual gripping, because the sliding of the jaw members 6a and 6b with respect to one another causes a rotation of the needle 9 around its longitudinal axis.

To minimize abrasion of the gripping surface of the slidable jaw member 6a or of the slidable portion 6c in the contact area with the surgical needle 9, at least the portion of the gripping surface of at least one jaw member 6a, 6b serving to receive a surgical needle 9 that is to be held consists of a hard metal.

Displacement of the slidable jaw member 6a or the slidable portion 6c of the jaw member 6a occurs via a power drive 10 that is positioned on the handle 5 and preferably is configured as an adjusting wheel 11 that can rotate around the longitudinal axis of the shaft 2.

The slidable jaw member 6a or the slidable portion 6c of the jaw member 6a and the power drive 10 are in operative connection with one another via an actuation element 12 that converts the rotary motion of the adjusting wheel 11 into a longitudinal movement of the slidable jaw member 6a or of the slidable portion 6c of the jaw member 6a.

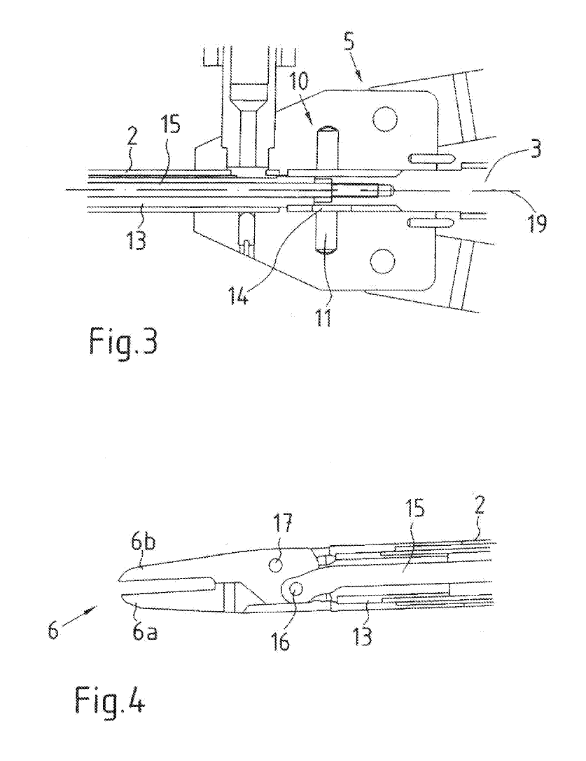

The embodiments shown in FIGS. 3 through 8 for configuring a medical needle holder 1 are distinguished from one another essentially in the fact that the pivoting of the pivotable jaw member 6b occurs via the pivotable gripping member 3 of the handle 5 and the displacement of the slidable jaw member 6a or of the slidable portion 6c of the jaw member 6a occurs via the power drive 10.

In the first embodiment of the medical needle holder 1 illustrated in FIGS. 3 and 4, the actuating element 12 and the push-pull element 8 are positioned coaxially to one another in the shaft 2.

As can be seen from FIG. 3, the actuating element 12 in this embodiment is configured as a hollow tube 13 positioned in the shaft 2, such that the adjusting wheel 11 and the hollow tube 13 are in operative connection with one another via a threading 14 in such a way that the rotary motion of the adjusting wheel 11 is transmitted into a purely axial displacement of the hollow tube 13.

The push-pull element 8 for actuating the pivotable jaw member 6b via the pivotable gripping member 3 is, in this embodiment, configured as a push-pull rod 15 that is positioned to slide axially in the hollow tube 13 and that is coupled on the proximal end in a friction locking with the pivotable gripping member 3 of the handle 5.

FIG. 4 shows the distal end of the medical needle holder 1 according to the previously described first embodiment. As can be seen from the illustration, the push-pull rod 15 is mounted on the pivotable jaw member 6a via a coupling point 16 in such a way that, upon an axial sliding of the push-pull rod 15 in the distal direction, the pivotable jaw member 6b is pivoted around a pivot axis 17 into the opened position shown in broken lines in FIG. 1. Conversely, the axial pulling back of the push-pull rod 15 in the proximal direction causes the pivotable jaw member 6b to be converted into the closed position.

According to the illustrated first embodiment, the slidable jaw member 6a constitutes the first end of the hollow tube 13, so that the rotation of the adjusting wheel 11 by transmission via the thread 14 causes a direct axial displacement of the slidable jaw member 6a. Alternatively to the illustrated embodiment, it is also possible for the distal end of the hollow tube 13 to be configured from a slidable portion 6c of the jaw member 6a.

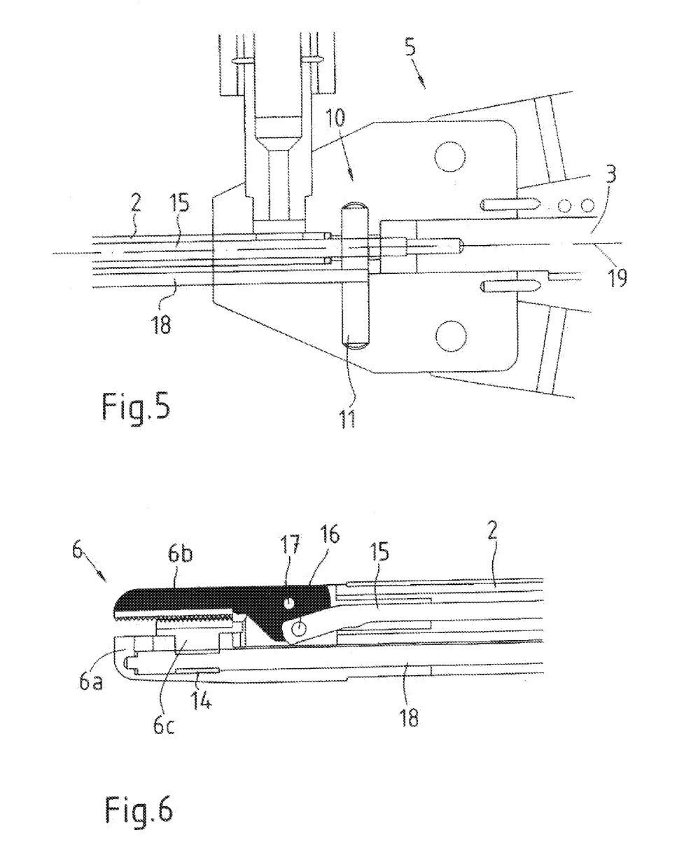

In the alternative embodiments illustrated in FIGS. 5 through 8 for configuring the relative slidability of the jaw members 6a and 6b of the tool 6, the entire jaw member 6a is not configured to be slidable in the longitudinal direction of the shaft 2, but only the portion 6c of the jaw member 6a that serves for clamping reception of the needle 9.

In the second embodiment of the medical needle holder 1 illustrated in FIGS. 5 and 6, the actuating element 12 and the push-pull element 8 are positioned parallel alongside one another in the shaft 2.

As can be seen from FIG. 5, the actuating element 12 is configured in this embodiment as a torsion bar 18 positioned in the shaft 2, such that the adjusting wheel 11 and the torsion bar 18 are in friction-locked connection with one another in such a way that the rotary motion of the adjusting wheel 11 is transmitted directly into a rotation of the torsion bar 18 around the longitudinal axis 19 of the shaft 2.

The push-pull element 8 for actuating the pivotable jaw member 6b via the pivotable gripping member 3 is, in this embodiment, configured as a push-pull rod 15 that is positioned to slide axially in the hollow tube 13 and that is coupled on the proximal end in friction-locked connection with the pivotable gripping member 3 of the handle 5.

FIG. 6 shows the distal end of the medical needle holder 1 according to the previously described second embodiment. As can be seen from the illustration, the push-pull rod 15 is positioned on the pivotable jaw member 6a via a coupling point 16 in such a way that the pivotable jaw member 6b, upon axial sliding of the push-pull rod 15 in the distal direction, is pivoted around a pivot axis 17 into the opened position shown in broken lines in FIG. 1. Conversely, the axial pulling back of the push-pull rod 15 in the proximal direction causes the pivotable jaw member 6b to be converted into the closed position.

According to the illustrated second embodiment, in this embodiment the entire jaw member 6a is not positioned to slide in the longitudinal direction of the shaft 2, but only a portion 6c of the jaw member 6a. In order to convert the rotary motion of the torsion bar 18, which was caused by rotation of the adjusting wheel 11, into a purely axial movement of the slidable portion 6c of the jaw member 6a, the distal end of the torsion bar 18 and the slidable portion 6c of the jaw member 6a are in operative connection with one another via a threading 14.

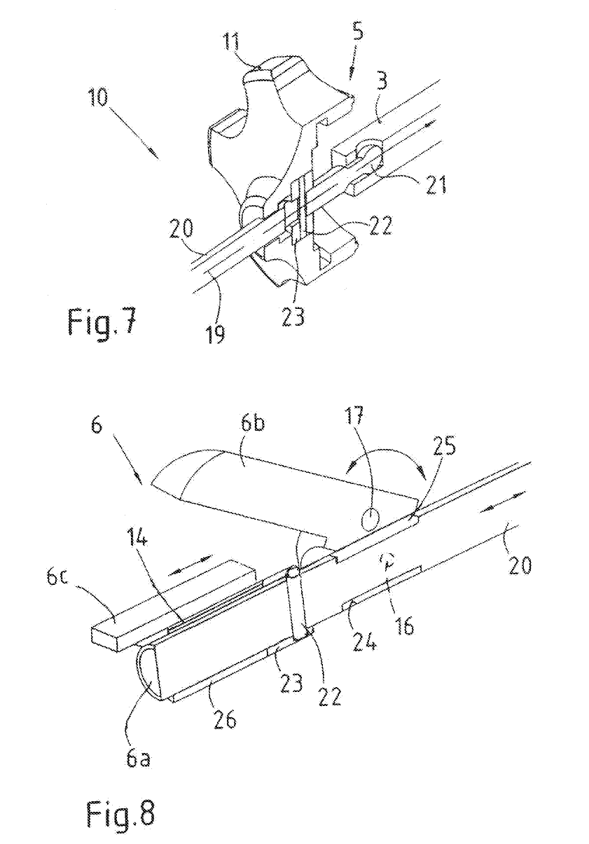

In the third embodiment of the medical needle holder 1 shown in FIGS. 7 and 8, the actuating element 12 and the push-pull element 8 are configured as a one-piece, rod-shaped drive element 20 positioned in the shaft 2.

As can be seen from FIG. 7, to transmit the axial motion from the movably configured gripping member 3 of the handle 5 onto the pivotable jaw member 6b of the tool 6, the drive element 20 is mounted on the proximal end on the movably configured gripping member 3 via a spherical connecting element 21, such that the spherical configuration of the proximal end of the drive element 20 makes possible a freely rotatable mounting of the drive element 20 on the movable gripping member 3, with simultaneous free play to transmit the axial motion.

As can be further seen from FIG. 7, the adjusting wheel 11 and the torsion bar 18 are in a friction-locked connection with one another in such a way that the rotary motion of the adjusting wheel 11 is transmitted directly into a rotation of the drive element 20 around the longitudinal axis 19 of the shaft 2, such that to configure the friction-locked connection of the adjusting wheel 11 with the drive element 20, a driving pin 22 is positioned in the drive element 20 to engage in the adjusting wheel 11.

To uncouple the axial motion and the rotary motion of the drive element 20 from one another, a groove-shaped equalizing guide 23 is configured in the adjusting wheel 11 in the area of the friction-locked connection of the drive element 20 with the adjusting wheel 11, so that the drive element 20 can be slid in the axial direction via the pivotable gripping member 3 without being blocked by the friction-locked coupling between the adjusting wheel 11 and the drive element 20.

FIG. 8 shows the distal end of the medical needle holder 1 according to the previously described third embodiment. As can be seen from the illustration, to transmit the axial motion from the movably configured gripping member 3 of the handle 5 onto the pivotable jaw member 6b of the tool 6, the drive element 20 on the distal end in the area of the mounting on the pivotable jaw member 6a involves a surrounding groove 24 being configured in which a ring 25 is positioned that can pivot around the longitudinal axis of the drive element 20 and on which the pivotable jaw member 6a is mounted via a coupling point 16 in such a way that the pivotable jaw member 6b upon axial sliding of the drive element 20 in the distal direction is pivoted around a pivot axis 17 into the opened position shown in broken lines in FIG. 1. Conversely, the axial pulling back of the drive element 20 in the proximal direction causes the conversion of the pivotable jaw member 6 into the closed position.

According to an illustrated third embodiment, in this embodiment as well, the entire jaw member 6a is not configured in the longitudinal direction of the shaft 2, but only a portion 6c of the jaw member 6a.

To transmit the rotary motion of the adjusting wheel 11 into a longitudinal movement of the slidable portion 6c of the jaw member 6a, the drive element 20 on the distal end in the area of the mounting on the slidable portion 6c of the jaw member 6a is in operative connection in friction-locking with a sheath 26 that is mounted coaxially on the distal end of the drive element 20 and that is connected via a threading 14 with the slidable portion 6c of the jaw member 6a, such that to configure the friction-locked connection of the adjusting wheel 11 with the drive element 20, a driving pin 22 is positioned in the drive element 20 to engage in the sheath 26.

To uncouple the axial motion and the rotary motion of the drive element 20 from one another, a groove-shaped equalizing guide 23 is configured in the sheath 26 in the area of the friction-locked connection of the drive element 20 with the sheath 26, so that the drive element 20 can be slid in the axial direction via the pivotable gripping member 3 without becoming blocked by the friction-locked coupling between the sheath 26 and the drive element 20.

The medical needle holder 1 as previously described and as illustrated in FIGS. 1 through 8 is operated as follows.

Starting from the opened position of the tool 6 illustrated in broken lines in FIG. 1, the needle holder 1 is placed by the operator in such a way that the surgical needle 9 comes to rest between the jaw members 6a and 6b of the tool 6. Then the operator presses the gripping members 3 and 4 of the handle 5 together, so that the pivotable jaw member 6b is closed and the needle is held clamped between the two jaw members 6a and 6b.

The needle 9, which as a rule is shaped in a sickle-like curvature, in order to suture a surgical site, must be aligned in the needle holder 1 in such a way that the operator can insert the needle 9 at the correct angle into the tissue that is to be sutured.

Alignment of the needle 9 held between the jaw members 6a and 6b occurs with the described needle holder 1 in such a way that, by actuating the power drive 10 positioned on the handle 5, either the entire jaw member 6a or only a portion 6c of the jaw member 6a is slid with respect to the other jaw member 6b in the longitudinal direction of the shaft 2, causing a rotation of the needle 9 around its longitudinal axis.

In order, on the one hand, to ensure a durably secure gripping of the surgical needle 9 in the tool 6 and, on the other hand, to relieve the operator, so that he/she is not required continually to actuate the gripping members 3 and 4 of the handle 5, the gripping members 3 and 4 of the handle 5 can be secured in their respective positions via a blocking mechanism 27, as can be seen from FIGS. 1 and 2.

By means of this blocking mechanism 27, it is possible to secure the needle between the jaw members 6a and 6b in a first incision only in such a way that the needle can still be rotated for alignment and only thereafter does the definitive clamping of the needle 9 in the correct position occur.

After releasing the surgical needle 9, in order to allow the slidable jaw member 6a or the slidable portion 6c of the jaw member 6a to resume the same freedom of movement for renewed grasping of the needle 9, the slidable jaw member 6a or the slidable portion 6c of the jaw member 6a is pre-tensed into a starting position by a spring element that is not illustrated.

A medical needle holder 1 of this configuration is distinguished by the fact that a simple positioning of the needle 9 in the tool 6 becomes possible without use of an additional instrument.

1. A medical needle holder comprising
a shaft,
a tool positioned on the distal end of the shaft having two jaw members, such that one jaw member of the tool is configured to pivot with respect to the other jaw member of the tool, and
a handle positioned on the proximal end of the shaft, and
at least two gripping members, such that the pivotable jaw member of the tool can be actuated by a movably configured gripping member of the handle,
wherein at least one portion of the non-pivotable jaw member of the tool is configured so that it can slide in relation to the other jaw member of the tool in the longitudinal direction of the shaft and
wherein the sliding in the longitudinal direction of the shaft occurs via a power drive positioned on the handle.

2. The medical instrument of claim 1, wherein the power drive is configured as an adjusting wheel.

3. The medical instrument of claim 2, wherein the slidable jaw member or the slidable portion of the jaw member and the power drive are in operative connection with one another via an actuation element, which converts the rotary motion of the adjusting wheel into a longitudinal movement of the slidable jaw member or of the slidable portion of the jaw member.

4. The medical instrument of claim 1, wherein the pivotable jaw member of the tool and the movably configured gripping member of the handle are in operative connection with one another via a push-pull element.

5. The A medical instrument of claim 3, wherein the actuation element and the push-pull element are positioned coaxially to one another in the shaft.

6. The medical instrument of claim 5, wherein the actuation element is configured as a hollow tube positioned in the shaft, such that the adjusting wheel and the hollow tube are in operative connection with one another via a threading and such that the slidable jaw member or the slidable portion of the jaw member forms the distal end of the hollow tube.

7. The medical instrument of claim 6, wherein the push-pull element is configured as a push-pull rod positioned to slide axially in the hollow tube.

8. The medical instrument of claim 3, wherein the actuation element and the push-pull element are positioned parallel alongside one another in the shaft .

9. The medical instrument of claim 8, wherein the actuation element is configured as a torsion bar positioned in the shaft, such that the adjusting wheel and the torsion bar are in friction-locked connection with one another and such that the slidable portion of the jaw member and the distal end of the torsion bar are in operative connection with one another via a threading.

10. The medical instrument of claim 9, wherein the push-pull element is configured as a push-pull rod positioned in the shaft.

11. The medical instrument of claim 3, wherein the actuation element and the push-pull element are configured as a one-piece rod-shaped drive element positioned in the shaft.

12. The medical instrument of claim 11, wherein to transmit the axial motion from the movably configured gripping member of the handle onto the pivotable jaw member of the tool, the drive element on the proximal end is mounted via a spherical connection element on the movably configured gripping member and on the distal end in the area of the mounting on the pivotable jaw member in the outer mantle surface of the drive element a surrounding groove is configured in which a ring is positioned that can rotate around the longitudinal axis of the drive element and on which the pivotable jaw member is mounted.

13. The medical instrument of claim 11, wherein to transmit the rotary motion of the adjusting wheel into a longitudinal movement of the slidable portion of the jaw member, the drive element is in a friction-locked connection with the adjusting wheel on the proximal end and on the distal end in the area of the mounting on the slidable portion of the jaw member is in friction-locked connection with a sheath that is mounted coaxially on the distal end of the drive element and that is connected with the slidable portion of the jaw member via a threading.

14. The medical instrument of claim 13, wherein in the areas of the friction-locked connections of the drive element with the adjusting wheel as well as of the drive element with the sheath, equalizing guides are configured to uncouple the axial motion and rotary motion of the working element from one another.

15. The medical instrument of claim 13, wherein to configure the friction-locked connections of the drive element with the adjusting wheel as well as of the drive element with the sheath, driver pins are positioned in the drive element to engage the adjusting wheel and sheath components with one another.

16. The medical instrument of claim 15, wherein the equalizing guides are configured as grooves positioned in the adjusting wheel and in the sheath and running in the axial direction, in which the driver pins are mounted.
